SEQUENCE LISTING (9 sequences)

SEQ ID NO: 1 - DNA, 33 bp, Streptococcus pneumoniae
aaacgtggatcccatatgaaagtcatagaagtg

SEQ ID NO: 2 - DNA, 30 bp, Streptococcus pneumoniae
aaacgtgaattcctaaatattataagctac

SEQ ID NO: 3 - DNA, 26 bp, Salmonella typhimurium
aaacgtcatatgattattactattcc

SEQ ID NO: 4 - DNA, 29 bp, Salmonella typhimurium
aaacgtgtcgacttaagaccaattccgag

SEQ ID NO: 5 - DNA, 23 bp, Staphylococcus aureus
agctcatatgaagctgatacgcg

SEQ ID NO: 6 - DNA, 25 bp, Staphylococcus aureus
agctgaattcttacacctgcccggc

SEQ ID NO: 7 - protein, 323 aa, Staphylococcus aureus
MKVIEVTHPIQSKQYITEDVAMAFGFFDGMHKGHDKVFDILNEIAEARSLKKAVMTFDPHPSVVLNPKRKRTTYLTPLSDKIEKISQHDIDYCIVVNFSSRFANVSVEDFVENYIIKNNVKEVIAGFDFTFGKFGKGNMTVLQEYDAFNTTIVSKQEIENEKISTTSIRQDLINGELQKANDALGYIYSIKGTVVQGEKRGRTIGFPTANIQPSDDYLLPRKGVYAVSIEIGTENKLYRGVANIGVKPTFHDPNKAEVVIEVNIFDFEDNIYGERVTVNWHHFLRPEIKFDGIDPLVKQMNDDKSRAKYLLAVDFGDEVAYNI

SEQ ID NO: 8 - protein, 305 aa, Streptococcus pneumoniae
MIITIPIKNQKDIGTPSDSVVVLGYFDGIHKGHQELFRVANKAARKDLLPIVVMTFNESPKIALEPYHPDLFLHILNPAERERKLKREGVEELYLLDFSSQFASLTAQEFFATYIKAMNAKIIVAGFDYTFGSDKKTAEDLKDYFDGEVIIVPPVEDEKGKISSTRIRQAILDGNVKEAGKLLGAPLPSRGMVVHGNARGRTIGYPTANLVLLDRTYMPADGVYVVDVEIQRQKYRAMASVGKNVTFDGEEARFEVNIFDFNQDIYGETVMVYWLDRIRDMTKFDSVDQLVDQLKADEEVTRNWS

SEQ ID NO: 9 - protein, 312 aa, Salmonella typhimurium
MKLIRGIHNLSQAPHGCVLTIGNFDGVHRGHRALLQGLQEEGRRRGLPVVVMIFEPQPLELFATDKAPARLTRLREKLHYLAQCGVDYVLCVKFDRRFAALTAQTFISELLVERLGVQFLAVGDDFRFGASRAGDFLLLQKAGAEYGFAVSSTQTFCEGGVRISSTAVRQALAEDNLALAESLLGHPFTISGRVVHGDELGRTIGFPTANLPLRRQVSPVKGVYAVEVTGLGDKPLPGVANIGTRPTVAGVRQQLEVHLLDVVMDLYGRHIDVILRKKIRNEQRFASLDELKAQIARDELTARKFFGLAGQV

(19) United States
(12) Patent Application Publication
(10) Pub. No.: US 2008/0274486 A1
(43) Pub. Date: Nov. 6, 2008

(54) Method of Assaying Fad Synthetase

(75) Inventor: David Ehmann, Arlington, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/571,843

(22) PCT Filed: Jul. 29, 2005
(86) PCT No.: PCT/GB05/02980

§ 371 Date: Jan. 9, 2007

Related U.S. Application Data
(60) Provisional application No. 60/592,689, filed on Jul. 30, 2004.

(51) Int. Cl. C12Q 1/48; C12Q 1/00

(52) U.S. Cl. 435/15; 435/4

(57) ABSTRACT

The present invention relates to methods of determining activity of flavin adenine dinucleotide (FAD) synthetase, and methods of identifying compounds that modulate the activity of this enzyme.

### FIELD OF THE INVENTION

The present invention relates to methods of determining the activity of flavin adenine dinucleotide (FAD) synthetase, and methods of identifying compounds that modulate the activity of this enzyme.

### BACKGROUND

FAD synthetase catalyzes the final steps in the biosynthesis of the cofactors flavin mononucleotide (FMN) and flavin adenine dinucleotide (FAD), which are essential cofactors for many enzymes.

FAD synthetase in bacteria is a bifunctional protein with two enzymatic activities. One enzymatic activity, flavokinase (FK) activity, is the phosphorylation of riboflavin (rfl) to produce FMN. The other enzymatic activity, FAD synthetase (FADS) activity, is the adenylation of FMN to produce FAD. This biosynthetic pathway has been predicted to be present in many pathogenic bacterial species and both enzymatic steps, FK and FADS, are predicted to be essential for bacterial viability (Gerdes et al., J. Bacteriol., 184: 4555-4572, 2002).

Studies investigating FAD synthetase enzymatic activity have been performed on a number of bacteria, including Bacillus subtilis (Kearney et al., J. Biol. Chem., 254:9551-9557, 1979) and Corynebacterium ammoniagenes (Spencer et al., Biochemistry, 15:1043-1053, 1976). FAD synthetase was shown to use ATP for FK and FADS activities in in vitro experiments for both Bacillus subtilis and Corynebacterium ammoniagenes. However, FAD synthetase enzymatic activity requires that its flavin substrate be in a particular redox state. For example, Bacillus subtilis was shown to require reduced flavins for FK and FADS activities in vitro. In contrast, FAD synthetase enzyme from Corynebacterium ammoniagenes has been reported to catalyze Rfl to FMN and FMN to FAD formation in the absence of added reducing agents and therefore uses the oxidized flavins as substrates.

### SUMMARY OF THE INVENTION

FAD synthetase is a bifunctional protein having two enzymatic activities: (1) flavokinase (FK) activity which is the phosphorylation of riboflavin to produce FMN, and (2) FAD synthetase (FADS) activity which is the adenylation of FMN to produce FAD, or the reversal of this reaction, the de-adenylation of FAD to FMN. The present invention is based, in part, on the finding that bacterial FAD synthetases from the pathogenic species Staphyloccus, Streptococcus, or Salmonella require a reduced substrate for their enzymatic activity. Moreover, it was found that bacterial FK from Salmonella requires reduced riboflavin for its activity.

The present invention also includes methods of identifying modulators of FADS activity by determining FMN or FAD formation or depletion in the presence of a test compound. In addition, modulators of FK activity can be determined by assaying for FMN formation in the presence of a test compound. The present invention is particularly suitable for identifying compounds of interest using high throughput screening (HTS).

Accordingly, in one aspect, the invention includes a method for determining the activity of a bacterial synthetase. The method includes contacting a bacterial FAD synthetase selected from the group consisting of Staphyloccus, Streptococcus, Salmonella, or a functional fragment thereof, with a reduced substrate of the FAD synthetase; and determining the activity of the bacterial FAD synthetase. The reduced substrate can be riboflavin, FMN or FAD.

In another aspect, the invention includes a method for determining the activity specifically of a FADS or bacterial flavokinase (FK). For example, the method includes contacting a bacterial FK selected from the group consisting of Staphyloccus, Streptococcus, Salmonella, or a functional fragment thereof, with a reduced riboflavin; and determining the activity of the bacterial FK.

In yet another aspect, the invention includes a method for identifying a compound capable of modulating bacterial flavin adenine dinucleotide (FAD) synthetase activity. The method includes contacting a bacterial FAD synthetase selected from the group consisting of Staphyloccus, Streptococcus, Salmonella, or a functional fragment thereof, with a reduced substrate of the bacterial FAD synthetase and a test compound; and determining the activity of the bacterial FAD synthetase wherein an increase or a decrease in activity in the presence of the compound compared to a control is indicative that the compound modulates FAD synthetase activity.

In yet another aspect, the invention includes a method for identifying a compound capable of modulating bacterial FADS or FK. For example, the method includes contacting a bacterial FK of Salmonella, or a functional fragment thereof, with a reduced riboflavin with a test compound; and determining the activity of the bacterial FK wherein an increase or a decrease in activity in the presence of the compound compared to a control is indicative that the compound modulates FK activity.

In the screening methods described above, the Staphyloccus can be S. aureus; the Streptococcus can be S. pneumoniae and Salmonella can be S. typhimurium. The test compounds used in the methods of the invention can be compounds such as peptides, peptidomimetics, small molecules, or other drugs.

### BRIEF DESCRIPTION OF THE FIGURES

FIG. 1 depicts a dendrogram representing phylogenetic distances of FAD synthetases.

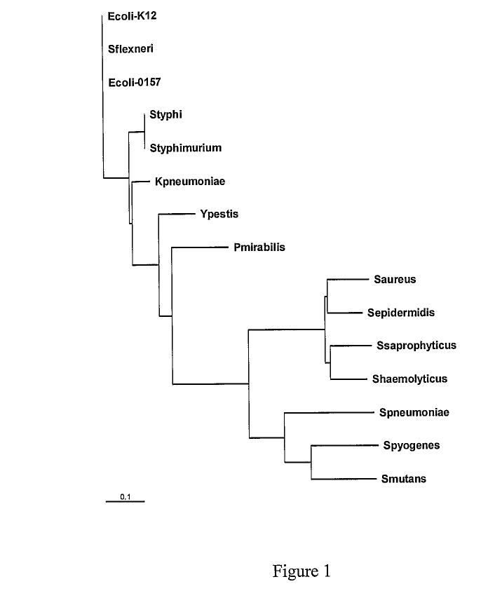

FIG. 2 depicts a protein alignment of FAD synthetases from Salmonella typhimurium, Staphylococcus aureus and Streptococcus pneumoniae.

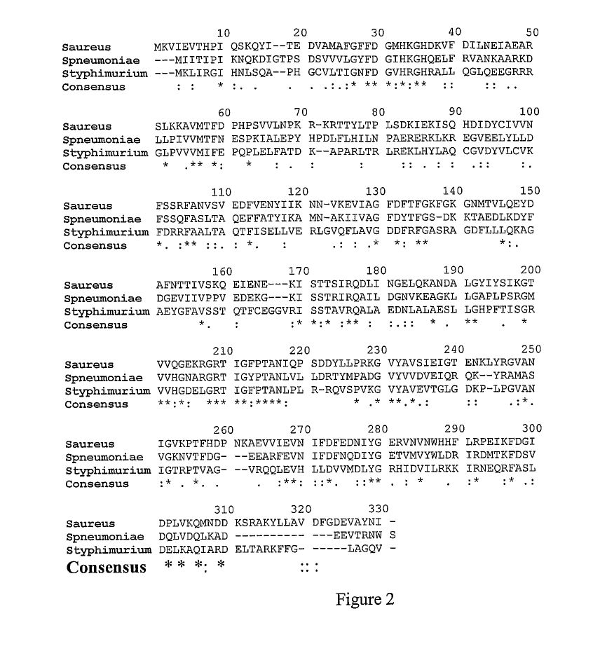

FIG. 3 depicts a compound having RibF modulating activity.

### DETAILED DESCRIPTION

The present invention provides, in part, methods for identifying novel antibacterial drugs. The present invention is based on the finding that the enzymatic activity of FAD synthetase from certain pathogens require a reduced substrate. The present invention provides methods for determining the enzymatic activity of FAD synthetase and for identifying modulators that increase or decrease its activity. Pathogens of interest include species from the family Enterobacteriaceae (such as Salmonella typhimurium), Staphylococcus species (such as Staphylococcus aureus, Staphylococcus epidermidis, Staphylococcus haemolyticus, and Staphylococcus saprophyticus), and Streptococcus species (such as Streptococcus pneumoniae, Streptococcus mutans, and Streptococcus pyogenes).

FAD Synthetase

The nucleic acids encoding FAD synthetase from various bacterial species are known in the art. For example, the nucleic acid sequence encoding FAD synthetase from Salmonella typhimurium is available in GenBank under AE008695; the nucleic acid sequence encoding FAD synthetase from Staphylococcus aureus is available in GenBank under NC_{—}002758; and the nucleic acid sequence encoding FAD synthetase from Streptococcus pneumoniae is available in GenBank under AE008474.

FAD synthetases from the bacterial genera of Staphyloccus, Streptococcus, or Salmonella can be readily obtained using methods well known in the art. For example, FAD synthetase from Salmonella typhimurium can be used to isolate cDNAs and genes encoding a homolog of FAD synthetase from the same class or other bacterial species. Examples of methods include, but are not limited to, methods of nucleic acid hybridization, and methods of DNA and RNA amplification as exemplified by various uses of nucleic acid amplification technologies (e.g., polymerase chain reaction (PCR) or ligase chain reaction).

For example, FAD synthetase, either as cDNAs or genomic DNAs, can be isolated directly by using all or a portion of the nucleic acid from Salmonella typhimurium as DNA hybridization probes to screen libraries from any desired bacterium employing methodology well known to those skilled in the art. Specific oligonucleotide probes based upon the FAD nucleic acid sequence of Salmonella typhimurium can be designed and synthesized. Moreover, the entire sequences can be used directly to synthesize DNA probes by methods known to the skilled artisan such as random primers, DNA labeling, nick translation, or end-labeling techniques. In addition, specific primers can be designed and used to amplify a part of, or full-length of, the instant sequences. The resulting amplification products can be labeled directly during amplification reactions or labeled after amplification reactions, and used as probes to isolate full length cDNA or genomic fragments under conditions of appropriate stringency. The sequence of the cDNA or genomic fragments can be obtained by sequencing these fragments. Methods for sequencing and characterizing an unknown gene based upon its homology to a known gene sequence are well known in the art (see, e.g., Sambrook et al., Molecular Cloning: A Laboratory Manual, CSH Press 1989).

Variants and Functional Fragments of FAD Synthetase

Variants of FAD synthetase include those variants that retain substantially the biological activity of FAD synthetase (e.g., FK and/or FADS activities). Typically variants retain more than 70% activity of the wild type FAD synthetase, for example, 75%, 80%, 85%, 90%, 95%, or 99% of the activity of the wild type FAD synthetase. The activity of the variant can be assayed using methods well known in the art. Variants include polypeptides that differ in amino acid sequence due to natural allelic variation or mutagenesis. In one example, functional variants typically contain only conservative variation or variation in non-critical residues or in non-critical regions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Since FAD synthetase is a bifunctional molecule having FK and FADS activities, functional fragments of FAD synthetase having these activities can be used in the methods of the invention. By functional fragment, it is a meant a fragment or variant of FAD synthetase, that has FK or FADS activity. Typically the FK or FADS fragment retains more than 70% activity of the wild type FAD synthetase, for example, 75%, 80%, 85%, 90%, 95%, or 99% of the activity of the wild type FAD synthetase.

Generally, FADS activity resides in the N-terminal domain and the FK activity resides in the C-terminal domain of FAD synthetase (Krupa et al., (2001) Trends Biochem. Sci., 10:1712-1728). For example, it has been shown that in E. coli, FAD synthetase amino acid mutations in the N-terminus of the protein greatly decreased FADS activity, while a mutation in the C-terminus of the protein decreased FK activity (see U.S. Pat. No. 5,514,574). Moreover, an X-ray crystal structure of FAD synthetase from the bacterium Thermotoga maritima (GenBank ID AAD35939) revealed a two-domain architecture with the N-terminal domain structurally homologous to adenylyltransferases and the C-terminal domain structurally homologous to flavin-binding proteins (Wang et al., (2003) Proteins, 52:633-635).

FADS or FK domains can be identified by means of routine computerized homology searching procedures. For example, homology studies can be used to predict domain boundaries for design of a fragment of a protein having FK activity or a fragment having FADS activity. A variety of known algorithms are disclosed publicly and a variety of commercially available software for conducting search means can be used. Examples of software include MacPattern (EMBL), BLASTN and BLASTX (NCBIA). For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify a FK domain or a FADS domain which are homologous to a known FK domain or FADS domain. In one example, BLAST nucleotide searches can be performed with the NBLAST program, score=100, wordlength=12 to obtain nucleotide sequences homologous FADS or FK domains. BLAST protein searches can be performed with the XBLAST program, score=50, wordlength=3 to obtain amino acid sequences homologous to FADS or FK domains. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

In one example, the FADS domain in S. aureus can be identified by comparing with Thermotoga maritima FADS structure (Wang et al. (2003) Proteins 52:633-635). For example, the N-terminal domain of Thermotoga maritima, residues Val₂ to Ser₁₃₆, can be used to predict a FADS domain boundary for the S. aureus enzyme. By comparing the N-terminal domain with the sequence of the S. aureus enzyme, proteins from amino acid Met₁ to approximately Ser₁₄₈ are predicted to have FADS activity and amino acid Ser₁₄₈ to Ile₃₂₃ of S. aureus are predicted to have FK activity.

The variants or predicted polypeptides can then be readily tested for their activities. Briefly, a polypeptide fragment predicted to have FADS activity may be tested for its ability to catalyze the formation of FAD from FMN as described in Efimov et al. (1998) Biochemistry 37:9716-9723, the contents of which are incorporated herein by reference, and a polypeptide fragment predicted to have FK activity could be tested for its ability to catalyze the formation of FMN from riboflavin in Efimov et al. (1998) Biochemistry 37:9716-9723, the contents of which are incorporated herein by reference.

FAD Synthetase Proteins

FAD synthetase proteins, or fragments having FADS activity, used in the method of the invention can be isolated from a bacterial species of interest. Methods of isolating FAD synthetase are known in the art, for example, see Efimov et al. (1998) Biochemistry 37:9716-9723, the contents of which are incorporated herein by reference. Alternatively, the FAD synthetase, or functional fragments thereof, can be produced recombinantly. If the FADS is produced recombinantly, the FAD synthetase can be cloned into a suitable expression vector. Typically, recombinant expression vectors include one or more regulatory sequences, selected on the basis of the host cells to be used for expression. The regulatory sequences are operatively linked to the FAD synthetase nucleic acid sequence to be expressed. “Operably linked” is intended to mean that the FAD synthetase nucleic acid sequence is linked to the regulatory sequence(s) in a manner which allows for expression of the FAD synthetase nucleic acid sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7.

The expression vector can then be introduced into a host cell of interest. For example, the host cell can be any prokaryotic or eukaryotic cell. For example, a functional fragment having FADS activity can be expressed in bacterial cells such as E. coli, insect cells, yeast or mammalian cells. Other suitable host cells are known to those skilled in the art. The expression vector can be introduced into the host cell via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

Reduced Substrates

The methods of the invention require providing a reduced flavin substrate for FADS or a reduced riboflavin for FK activity.

To provide a reduced flavin, a reducing agent can be added to a solution of oxidized flavin to convert it to its reduced form. The reducing agent can be an enzymatic system, for example, an oxidoreductase protein and nicotinamide substrate, such as the nicotinamide adenine dinucleotide phosphate NAD(P)H-dependent oxidoreductase from the bacterium Vibrio harveyi (Tu, Antiox. Redox. Signal., 3:881-897, 2001). Alternatively, the reducing agent can be a chemical, such as sodium dithionite, borohydrides such as but not limited to sodium borohydride, or hydrogen in the presence of a catalyst such as but not limited to palladium (Ghisla, Methods Enzymol., 66:361-373, 1980). Suitable reducing chemicals can be readily identified by those skilled in the art.

The efficacy of a reducing agent can be tested for its ability to generate substrates for FADS activity. For example, to perform such an experiment, reactions are performed at 25° C. in the presence of the test reducing agent, 25 mM Tris.HCl pH 7.5, ATP, riboflavin, and FAD synthetase. After 15 min incubation, reactions are quenched and analyzed to quantify the amount of, for example, FAD produced.

Measuring FAD Synthetase or FK Activity

FAD synthetase enzymatic activity, i.e., FADS or FK activity, can be measured by quantifying the amount of substrate (for example, riboflavin, ATP, or FMN) turnover in in vitro reactions or product formation (for example, ADP, FMN, PPi, or FAD). Such methods are known in the art. For example, substrate depletion and/or production can be measured directly, for example, by chromatographic separation with concomitant absorbance or fluorescence detection of each individual flavin. In addition, FMN and FAD can be measured by coupling to an enzyme that requires FMN or FAD for turnover.

Briefly, in one example, to determine FADS activity the amount of FMN being depleted or the FAD being produced can be determined by analyzing the forward reaction. In this example, an in vitro enzymatic reaction can be performed by initiating the FADS reaction by addition of FMN. After quenching the reaction, the amount of FMN depleted or FAD produced can be quantified by anion exchange high-performance liquid chromatography (Entsch et al. (1983) Anal. Biochem. 13:401-408, the contents of which are incorporated herein by reference).

Alternatively, the reverse reaction where FADS catalyzes FMN formation from FAD can be determined. Briefly, in this example, one can perform an in vitro enzymatic reaction by initiating the FADS reaction by addition of FAD. After quenching the reaction, the amount of FAD depleted or FMN produced can be quantified by anion exchange high-performance liquid chromatography (Entsch et al. (1983) Anal. Biochem. 13:401-408).

In another example, FMN and FAD can be measured by coupling to an enzyme that requires FMN or FAD. An example of a FMN coupling enzyme is Vibrio fischeri luciferase (Stanley (1971) Anal. Biochem. 39: 441-453). Using Vibrio fischeri luciferase, the luciferase activity is quantified by the amount of luminescence produced. An example of a FAD coupling enzyme is apo-D-amino acid oxidase (Hinkkanen (1983) Anal. Biochem. 132: 202-208). The activity of the apo-D-amino acid oxidase can be quantified by the amount of hydrogen peroxide produced.

For FK activity, the amount of riboflavin depleted or FMN produced can be determined. In this example, an in vitro enzymatic reaction can be performed by initiating the FK reaction by addition of riboflavin. After quenching the reaction, the amount of riboflavin depleted or FMN produced can be quantified by anion exchange high-performance liquid chromatography (Entsch et al. (1983) Anal. Biochem. 13:401-408).

Alternatively, the determination of the depletion of the substrate ATP or the production of the products ADP and pyrophosphate can be used to determine FAD synthetase enzymatic activity. For the FK reaction, the amount of ATP depleted or ADP produced can be determined by anion exchange high-performance liquid chromatography. For the FK reaction, the amount of ADP produced can be determined by coupling to an enzyme system that requires ADP for turnover such as pyruvate kinase and lactate dehydrogenase where the lactate dehydrogenase activity is quantified by the amount of NADH consumed (Jaworek et al. (1985) in Methods of Enzymatic Analysis (Bergmeyer, H. U., ed) 3^{rd} Ed, 7:365-369). For the FADS reaction, the amount of pyrophosphate produced by addition of pyrophosphatase enzyme and detection of released phosphate can be used to determine FADS activity. In one example the amount of phosphate can be quantified by the use of malachite green and ammonium molybdate, which gives a colorimetric readout (Lanzetta et al. (1979) Anal. Biochem. 100: 95-97).

Screening Methods

The invention includes methods for identifying a compound capable of modulating bacterial flavin adenine dinucleotide (FAD) synthetase activity. In one example, the method includes contacting a bacterial FAD synthetase selected from the group consisting of Staphyloccus, Streptococcus, Salmonella, or a functional fragment thereof, with a reduced substrate of the bacterial FAD synthetase and a test compound; and determining the activity of the bacterial FAD synthetase wherein an increase or a decrease in activity in the presence of the compound compared to a control is indicative that the compound modulates FAD synthetase activity. The activity of the bacterial FAD synthetase can be determined as described above.

In another example, the invention includes a method for identifying a compound capable of modulating bacterial FADS or FK. The method can include contacting a bacterial FK of Salmonella, or a functional fragment thereof, with a reduced riboflavin and a test compound; and determining the activity of the bacterial FK wherein an increase or a decrease in activity in the presence of the compound compared to a control is indicative that the compound modulates FK activity.

The test compounds used in the methods described above include compounds such as peptides, peptidomimetics, small molecules, or other drugs.

The foregoing examples are meant to illustrate the invention and are not to be construed to limit the invention in any way. Those skilled in the art will recognize modifications that are within the spirit and scope of the invention.

### EXAMPLES

### Example 1

### Isolation of the S. aureus, S. pneumoniae, and S. typhimurium ribF Genes

To clone ribF, PCR forward and reverse primers were designed with an Nde1 site (underlined) flanking the 5′ end of the gene and a Sal1 or EcoR1 site (underlined) flanking the 3′ end. DNA was amplified from S. aureus (5′AAACGTGGATCCCATATGAAAGTCATAGAAGTG 3′ (SEQ ID NO:1)) and (5′AAACGTGAATTCCTAAATATTATAAGCTAC3′(SEQ ID NO:2)), S. pneumoniae (5′AAACGTCATATGATTATTACTATTCC3′ (SEQ ID NO:3)) and (5′AAACGTGTCGACTTAAGACCAATTCCGAG3′(SEQ ID NO:4)), and S. typhimurium (5′AGCTCATATGAAGCTGATACGCG3′ (SEQ ID NO:5)) and (5′AGCTGAATTCTTACACCTGCCCGGC3′(SEQ ID NO:6)) using High Fidelity PCR Master polymerase (Roche, Ind.) as described by manufacturer.

The PCR products were digested with Nde1 and Sal1 and ligated into an expression vector cut with both restriction enzymes. The ligation mixtures were used to transform chemically TOP10 competent cells. Cells were plated onto LB plates supplemented with kanamycin (25 ug/ml) and incubated at 37° C. overnight. Plasmids prepared from different bacterial colonies were checked for the correct insert by DNA sequencing. The correct plasmids were transformed into E. coli for protein overexpression to generate a strain for S. aureus RibF, for S. pneumoniae RibF, and for S. typhimurium RibF.

(1) Purification of the S. aureus, S. pneumoniae, and S. typhimurium RibF Proteins

To purify S. aureus RibF, cells of E. coli were grown at 37° C. in LB media with 25 μg/mL kanamycin until an OD₆₀₀ of 0.5, then induced with 0.5 mM IPTG and grown at 20° C. for 4 hr. Cells from 6 L of this fermentation were resuspended in 70 mL of lysis buffer: 50 mM Tris.HCl pH 8.0, 1 mM DTT, 10 mM EDTA, 25 mM NaCl. Cells were broken by French press at 18,000 psi, twice, at 4° C. then centrifuged at 10,000 rpm at 4° C. for 60 min. The supernatant fraction was applied on to a 20 mL Q-Seph FF (Amersham Biosciences), equilibrated with buffer A: 50 mM Tris.HCl pH 8.0, 1 mM DTT, 1 mM EDTA, 25 mM NaCl. The column was washed with 6 column volumes of buffer A and eluted with 10 column volumes of a linear gradient from 25 mM to 350 mM NaCl. The main fraction was pooled and adjusted to 1 M ammonium sulfate. This fraction was loaded on to 20 mL Ph-Sepharose column (Amersham Biosciences, NJ), equilibrated with buffer: 50 mM Tris.HCl pH 8.0, 1 mM DTT, 1 mM EDTA, 1 M ammonium sulfate. The protein was eluted with a linear gradient from 1 M to 0 M ammonium sulfate. Fractions containing protein were pooled, concentrated in Amicon CentriPrep concentrator and applied to 200 mL HiPrep Sephacryl S-200 (Amersham Biosciences, NJ) equilibrated with 25 mM Tris.HCl pH 8.0, 150 mM NaCl, 10% glycerol, 1 mM DTT, 1 mM EDTA. Pure protein was aliquoted and stored at −80° C.

The same procedure as above was used to purify S. pneumoniae RibF.

To purify S. typhimurium RibF, E. coli cells were grown at 37° C. in LB media with 25 μg/mL kanamycin until an OD₆₀₀ of 0.5, then induced with 0.5 mM IPTG and grown at 20° C. for 3 hr. Cells from 2 L of this fermentation were resuspended in 75 mL of lysis buffer: 20 mM Tris.HCl pH 8.0, 1 mM DTT, 10 mM EDTA, 25 mM NaCl. Cells were broken by French press at 18,000 psi, twice, at 4° C. then centrifuged at 10,000 rpm at 4° C. for 30 min. The supernatant fraction was applied on to a 15 mL SP-Seph FF (Amersham Biosciences, NJ), equilibrated with buffer A: 20 mM Tris.HCl pH 8.0, 1 mM DTT, 1 mM EDTA, 25 mM NaCl. The column was washed with 3 column volumes of buffer A and eluted with 20 column volumes of a linear gradient from 25 mM to 500 mM NaCl. Fractions containing protein were pooled, concentrated in Amicon CentriPrep concentrator and applied to 200 mL HiPrep Sephacryl S-200 (Amersham Biosciences, NJ) equilibrated with 25 mM Tris.HCl pH 8.0, 150 mM NaCl, 10% glycerol, 1 mM DTT, 1 mM EDTA. Pure protein was aliquoted and stored at −80° C.

### Example 2

### Enzymatic Assay of the S. aureus, S. pneumoniae, and S. typhimurium RibF Proteins

To assay S. aureus RibF in vitro time course experiments were performed and analyzed by high-performance liquid chromatography. Reactions were carried out in 100 μL of 50 mM sodium phosphate pH 7.0, 0.002% Brij-35, 1 mM magnesium chloride, and 0.5 mM ATP. Where indicated, S. aureus RibF was added to 200 mM final concentration from a stock solution of 60 μM prepared as described above. Where indicated, sodium dithionite (Aldrich, Wis.) was added to 1 mM final concentration from a stock solution of 10 mM prepared in water bubbled with nitrogen. Where indicated, riboflavin was added to 0.050 mM from a stock solution of 0.10 mM prepared in water. Where indicated, FMN was added to 0.050 mM from a stock solution of 0.10 mM prepared in water. Reactions were incubated at 25° C. and quenched at varying times with 100 μL of 20 mM EDTA pH 8.0. Samples were analyzed by high-performance liquid chromatography (HPLC) by injecting 25 μL onto a quaternary amine anion exchange column 402.IC×250 mm (Vydac, CA) equilibrated with sodium phosphate pH 2.8. Peaks were identified by fluorescence detection (442 nm excitation wavelength, 520 emission wavelength) and comparison of retention times and areas to

TABLE 1

| Conditions | μM Riboflavin | μM FMN | μM FAD |
|---|---|---|---|
| No enzyme + riboflavin | 86 | 0 | 0 |
| S. aureus RibF + | 50 | 22 | 0 |
| riboflavin | | | |
| S. aureus RibF + sodium | 1 | 41 | 28 |
| dithionite + riboflavin | | | |
| S. aureus RibF + FMN | 0 | 44 | 0 |
| S. aureus RibF + sodium | 0 | 11 | 28 |
| dithionite + FMN | | | |

The data in Table 1 display that in the absence of sodium dithionite S. aureus RibF demonstrated detectable and measurable FK activity but not detectable and measurable FADS activity. In the presence of sodium dithionite S. aureus RibF demonstrated detectable and measurable FK and FADS activities.

To assay S. pneumoniae RibF in vitro time course experiments were performed and analyzed by high-performance liquid chromatography as described above except, where indicated, S. pneumoniae RibF was added to 200 nM final concentration from a stock solution of 6.0 μM prepared as described above. Data for the 45-minute time point are shown in Table 2.

TABLE 2

| Conditions | μM Riboflavin | μM FMN | μM FAD |
|---|---|---|---|
| No enzyme + riboflavin | 77 | 0 | 0 |
| S. pneumoniae RibF + | 17 | 53 | 0 |
| riboflavin | | | |
| S. pneumoniae RibF + | 27 | 25 | 16 |
| sodium dithionite + | | | |
| riboflavin | | | |
| S. pneumoniae RibF + FMN | 0 | 42 | 0 |
| S. pneumoniae RibF + | 0 | 13 | 26 |
| sodium dithionite + FMN | | | |

The data in Table 2 display that in the absence of sodium dithionite S. pneumoniae RibF demonstrated detectable and measurable FK activity but not detectable and measurable FADS activity. In the presence of sodium dithionite S. pneumoniae RibF demonstrated detectable and measurable FK and FADS activities.

To assay S. typhimurium RibF in vitro time course experiments were performed and analyzed by high-performance liquid chromatography as described above except, where indicated, S. typhimurium RibF was added to 1.3 μM final concentration from a stock solution of 26 μM prepared as described above. Data for the 10-minute time point are shown in Table 3.

TABLE 3

| Conditions | μM Riboflavin | μM FMN | μM FAD |
|---|---|---|---|
| No ATP + riboflavin | 36 | 0 | 0 |
| S. typhimurium RibF + | 36 | 0 | 0 |
| riboflavin | | | |
| S. typhimurium RibF + sodium | 1 | 13 | 25 |
| dithionite + riboflavin | | | |

The data in Table 3 display that in the absence of sodium dithionite S. typhimurium RibF did not demonstrate detectable and measurable FK activity nor detectable and measurable FADS activity. In the presence of sodium dithionite S. typhimurium RibF demonstrated detectable and measurable FK and FADS activities.

### Example 3

### Preparation of a Functional Fragment of S. aureus RibF

A S. aureus flavokinase domain protein was designed by comparison to the T. maritima structure. The domain boundary was selected between Lys₁₄₆ and Ile₁₄₇. Because the C-terminal flavokinase domain protein was predicted to begin with an α-helix structural motif, the construct design was altered to include two mutations, T150D and S151E, that were predicted to provide stabilization of the N-terminal α-helix (Seale et al., (1994) Prot. Sci. 3:1741-1745).

A plasmid containing DNA encoding the S. aureus FK domain was prepared using standard molecular biology techniques similar to those described above for the full-length S. aureus protein. After overexpression in E. coli, the protein was purified using standard protein purification techniques similar to those described above for the full-length S. aureus protein.

The FK domain protein was assayed by detecting FMN formation using the HPLC assay described above for full-length S. aureus FAD synthetase. Reactions were carried out in 100 μL of 50 mM HEPES pH 7.5, 0.002% Brij-35, 10 mM magnesium chloride, 5.0 mM ATP, 20 μM riboflavin, and 50 nM enzyme. Similar rates of FK activity were seen with the FK domain protein compared to the full-length S. aureus FAD synthetase (Table 4).

TABLE 4

| Protein | FK Activity, min-1 |
|---|---|
| S. aureus FK domain | 1.6 |
| S. aureus full-length RibF | 1.7 |

### Example 4

### Identification of a Compound that Modulates RibF Activity

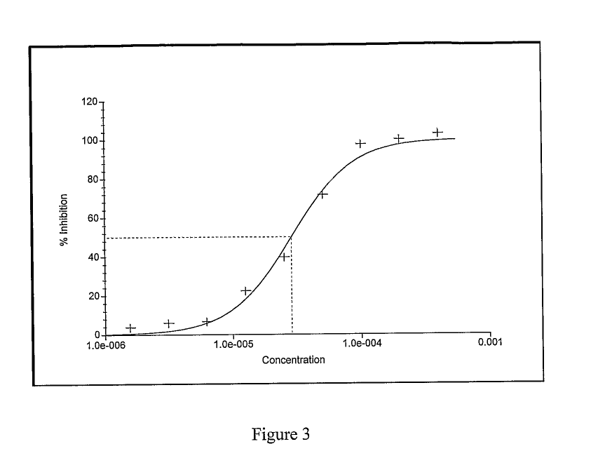

Assays of S. aureus RibF were performed in the presence of increasing amounts of Compound A to measure the amount of inhibition by Compound A. Both FK and FADS activities were assayed concomitantly by initiating reactions with riboflavin detecting pyrophosphate product. Reactions were carried out at room temperature in a 96-well microtiter plate containing 100 μL of 50 mM HEPES pH 7.5, 0.002% Brij-35, 1 mM ethylenediamine tetraacetic acid, 0.1 mg/mL pyrophosphatase, 2% dimethyl sulfoxide, 2 mM magnesium chloride, 100 μM ATP, 10 μM riboflavin, 10 mM sodium dithionite, 80 nM S. aureus RibF, and Compound A varying from 0.78 to 400 μM. Reactions were quenched after 20 minutes with 10 μL 200 mM ethylenediamine tetraacetic acid. After 2 hours 150 μL of Malachite Green phosphate detection reagent (Lanzetta et al. (1979) Anal. Biochem. 100: 95-97) was added. Percent inhibition was calculated by comparison to reactions with no Compound A (no inhibition) and reactions with 20 mM ethylenediamine tetraacetic acid (full inhibition). As seen in FIG. 3, Compound A displayed dose-dependent inhibition with a concentration yielding 50% inhibition (IC₅₀) of 29 μM.

1. A method for determining the activity of a bacterial flavin adenine dinucleotide (FAD) synthetase comprising:
contacting a bacterial FAD synthetase selected from the group consisting of Staphyloccus, Streptococcus, Salmonella, or a functional fragment thereof, with a reduced substrate of the bacterial FAD synthetase; and
determining the activity of the bacterial FAD synthetase.

2. The method of claim 1, wherein the Staphyloccus is Staphyloccus aureus.

3. The method of claim 1, wherein the Streptococcus is Streptococcus pneumoniae.

4. The method of claim 1, wherein the Salmonella is Salmonella typhimurium.

5. The method of claim 1, wherein the reduced substrate is FMN or FAD.

6. A method for determining the activity of a bacterial flavokinase (FK) comprising;
contacting a bacterial FK of Salmonella, or a functional fragment thereof, with a reduced riboflavin; and
determining the activity of the bacterial FK.

7. The method of claim 6, wherein the Salmonella is Salmonella typhimurium.

8. A method for identifying a compound capable of modulating bacterial flavin adenine dinucleotide (FAD) synthetase activity comprising
contacting a bacterial FAD synthetase selected from the group consisting of Staphyloccus, Streptococcus, Salmonella, or a functional fragment thereof, with a reduced substrate of the bacterial FAD synthetase and a test compound; and
determining the activity of the bacterial FAD synthetase wherein an increase or a decrease in activity in the presence of a compound compared to a control is indicative that the compound modulates FAD synthetase activity.

9. The method of claim 8, wherein the Staphyloccus is Staphyloccus aureus.

10. The method of claim 8, wherein the Streptococcus is Streptococcus pneumoniae.

11. The method of claim 8, wherein the Salmonella is Salmonella typhimurium.

12. The method of claim 8, wherein the reduced substrate is FMN or FAD.

13. A method for identifying a compound capable of modulating bacterial flavokinase comprising:
contacting a bacterial FK of Salmonella, or a functional fragment thereof, with a reduced riboflavin and a test compound; and
determining the activity of the bacterial FK wherein an increase or a decrease in activity in the presence of a compound compared to a control is indicative that the compound modulates FK activity.

14. The method of claim 13, wherein the Salmonella is Salmonella typhimurium.
